SEQUENCE LISTING (36 sequences)

SEQ ID NO: 1 - DNA, 29 bp, Artificial Sequence
  Description of Artificial Sequence Synthetic primer
atcgagctcaaccatcatcgatgaattgc

SEQ ID NO: 2 - DNA, 25 bp, Artificial Sequence
  Description of Artificial Sequence Synthetic primer
tctggtaccctgaagcttgcatgcc

SEQ ID NO: 3 - DNA, 23 bp, Artificial Sequence
  Description of Artificial Sequence Synthetic primer
atagagctcccaccagataacgg

SEQ ID NO: 4 - DNA, 27 bp, Artificial Sequence
  Description of Artificial Sequence Synthetic primer
gcgctcgagtttcataagactttctcc

SEQ ID NO: 5 - DNA, 26 bp, Artificial Sequence
  Description of Artificial Sequence Synthetic primer
gacctcgagctggtttaatcttgccg

SEQ ID NO: 6 - DNA, 25 bp, Artificial Sequence
  Description of Artificial Sequence Synthetic primer
cgtggtaccttctttcagcatttcg

SEQ ID NO: 7 - DNA, 43 bp, Artificial Sequence
  Description of Artificial Sequence Synthetic primer
accgtggcggggatcttaagacccactttcacatttaagttgt

SEQ ID NO: 8 - DNA, 40 bp, Artificial Sequence
  Description of Artificial Sequence Synthetic primer
cgactctagaggatcctaagcacttgtctcctgtttactc

SEQ ID NO: 9 - DNA, 25 bp, Artificial Sequence
  Description of Artificial Sequence Synthetic primer
acggagctcatcttgataacgcgat

SEQ ID NO: 10 - DNA, 29 bp, Artificial Sequence
  Description of Artificial Sequence Synthetic primer
tacctcgagcatggaagtacctataattg

SEQ ID NO: 11 - DNA, 35 bp, Artificial Sequence
  Description of Artificial Sequence Synthetic primer
ggataaaccgtgtccctcgagcagcagtaatctcg

SEQ ID NO: 12 - DNA, 28 bp, Artificial Sequence
  Description of Artificial Sequence Synthetic primer
actggtacctctagaagttcatcaggcc

SEQ ID NO: 13 - DNA, 27 bp, Artificial Sequence
  Description of Artificial Sequence Synthetic primer
tgagagctcagagcaggaagtgaaagc

SEQ ID NO: 14 - DNA, 46 bp, Artificial Sequence
  Description of Artificial Sequence Synthetic primer
cctttttaccttagccagtttgtacatttcatggttctccatctcc

SEQ ID NO: 15 - DNA, 23 bp, Artificial Sequence
  Description of Artificial Sequence Synthetic primer
acaaactggctaaggtaaaaagg

SEQ ID NO: 16 - DNA, 28 bp, Artificial Sequence
  Description of Artificial Sequence Synthetic primer
agtggtaccgactgagtaatttggtgcg

SEQ ID NO: 17 - DNA, 27 bp, Artificial Sequence
  Description of Artificial Sequence Synthetic primer
atagagctcaaatcgagcaacggatcg

SEQ ID NO: 18 - DNA, 46 bp, Artificial Sequence
  Description of Artificial Sequence Synthetic primer
cggcaagattaaaccagtttgtacagtttcataagactttctccag

SEQ ID NO: 19 - DNA, 46 bp, Artificial Sequence
  Description of Artificial Sequence Synthetic primer
ctggagaaagtcttatgaaactgtacaaactggtttaatcttgccg

SEQ ID NO: 20 - DNA, 31 bp, Artificial Sequence
  Description of Artificial Sequence Synthetic primer
tcaggtaccaatgttttgatcaaacagaggg

SEQ ID NO: 21 - DNA, 22 bp, Artificial Sequence
  Description of Artificial Sequence Synthetic primer
attgagctcggttaccgtcgcc

SEQ ID NO: 22 - DNA, 32 bp, Artificial Sequence
  Description of Artificial Sequence Synthetic primer
gccattcgccttctcgagcacgacattcctcc

SEQ ID NO: 23 - DNA, 32 bp, Artificial Sequence
  Description of Artificial Sequence Synthetic primer
ggaggaatctcgtgctcgagaaggcgaatggc

SEQ ID NO: 24 - DNA, 24 bp, Artificial Sequence
  Description of Artificial Sequence Synthetic primer
agcggtaccacagttgatgcaacc

SEQ ID NO: 25 - DNA, 32 bp, Artificial Sequence
  Description of Artificial Sequence Synthetic primer
aaaaaaggtaccgcgttgaaagtgttgatctg

SEQ ID NO: 26 - DNA, 41 bp, Artificial Sequence
  Description of Artificial Sequence Synthetic primer
ttattcgtcgtgttcgtttaattgtcccgtagtcatattac

SEQ ID NO: 27 - DNA, 41 bp, Artificial Sequence
  Description of Artificial Sequence Synthetic primer
gaacacgacgaataactcgagtgtaaatgccgaatcaagcg

SEQ ID NO: 28 - DNA, 32 bp, Artificial Sequence
  Description of Artificial Sequence Synthetic primer
aaaaaagagctccagatcaatggtgcctttgg

SEQ ID NO: 29 - DNA, 32 bp, Artificial Sequence
  Description of Artificial Sequence Synthetic primer
tttggtaccgcattctgtttgctcagactatg

SEQ ID NO: 30 - DNA, 41 bp, Artificial Sequence
  Description of Artificial Sequence Synthetic primer
ttattcccactcttgaattgctcctttagagatgagtagtg

SEQ ID NO: 31 - DNA, 39 bp, Artificial Sequence
  Description of Artificial Sequence Synthetic primer
caagagtgggaataactcgagcctactccaaactcccgg

SEQ ID NO: 32 - DNA, 34 bp, Artificial Sequence
  Description of Artificial Sequence Synthetic primer
ttttttgagctcgtttctgcaaatcgaccttctg

SEQ ID NO: 33 - DNA, 30 bp, Artificial Sequence
  Description of Artificial Sequence Synthetic primer
cgcctcgagggttagctccgaagcaaaagc

SEQ ID NO: 34 - DNA, 33 bp, Artificial Sequence
  Description of Artificial Sequence Synthetic primer
cgcggtaccaatgctctcctgataatgttaaac

SEQ ID NO: 35 - DNA, 63 bp, Artificial Sequence
  Description of Artificial Sequence Synthetic primer
accgtcagggttaactgcagaaataggagataaaatatatggaccgcattattcaatcaccgg

SEQ ID NO: 36 - DNA, 41 bp, Artificial Sequence
  Description of Artificial Sequence Synthetic primer
tgcctctagcacgcgtttattcccactcttgcaggaaacgc

(19) United States
(12) Patent Application Publication
(10) Pub. No.: US 2015/0232805 A1
(43) Pub. Date: Aug. 20, 2015

(54) STRAINS AND METHODS FOR PLASMID MAINTENANCE

(71) Applicant: GLYCOS BIOTECHNOLOGIES, INC., Houston, TX (US)

(72) Inventors: Paul Campbell, Houston, TX (US); Ryan W. Black, Spring, TX (US); Stephanie Doneske, Katy, TX (US); Mai Li, Katy, TX (US); Daniel J. Monticello, The Woodlands, TX (US)

(21) Appl. No.: 14/601,639

(22) Filed: Jan. 21, 2015

Related U.S. Application Data
(63) Continuation of application No. PCT/US2013/051785, filed on Jul. 24, 2013.
(60) Provisional application No. 61/741,716, filed on Jul. 26, 2012.

(51) Int. Cl. C12N 1/20; C12N 1/32; C12N 15/70

(52) U.S. Cl. CPC C12N 1/20; C12N 15/70; C12N 1/32

(57) ABSTRACT

The present invention provides a host microorganism with a deletion, disruption or mutation in one or more enzymes of a glycerol dissimilation pathway and a plasmid without an antibiotic resistance gene but carrying one or more genes encoding enzymes involved in glycerol dissimilation, wherein the plasmid is stably maintained by the host microorganism when cultured on glycerol as a carbon source. Such a plasmid maintenance system is beneficial in applications where the presence of an antibiotic resistance gene or the use of antibiotics is either prohibited or problem.

### RELATED APPLICATIONS

This application is a continuation application of International Application No. PCT/US2013/051785, which designated the United States and was filed on Jul. 24, 2013, published in English, which claims the benefit of U.S. Provisional Application No. 61/741,716, filed on Jul. 26, 2012. The entire teachings of the above applications are incorporated herein by reference.

### FIELD OF THE INVENTION

The present disclosure generally relates to the use of a non-naturally occurring microorganism bearing a plasmid that is stably maintained in the presence of glycerol as a carbon source. More specifically, the plasmid may be stably maintained even though it does not contain an antibiotic resistance gene.

### BACKGROUND OF THE INVENTION

Plasmids are small, DNA-based molecules that are capable of replicating inside a host cell independently from chromosomal DNA. Plasmids have become important tools in medical and industrial biotechnology because they allow the introduction of foreign genes or additional copies of native genes. With the proper genetic elements, the expression of the introduced gene(s) can be controlled through the use of appropriate signals, typically a change in culture conditions, such as a heat shock, or through the addition of a chemical (inducer) such as isopropyl β-D-1-thiogalactopyranoside. Furthermore, many plasmids carry antibiotic resistance genes. The use of antibiotics and antibiotic resistance genes greatly facilitates the genetic manipulation of microorganisms in the research laboratory but creates problems in commercial processes. The presence of an antibiotic resistance gene in expression vectors greatly facilitates plasmid construction by providing a simple method of selecting for transformed bacteria carrying the expression vector. Furthermore, the continued presence of the antibiotic forces the microorganism to maintain the expression vector during its use, for example, for the production of a recombinant protein.

However, in many commercial applications, either the antibiotic or the antibiotic resistance gene is either undesirable or absolutely prohibited. It is economically impractical to use antibiotics in large-scale fermentations for the production of biofuels or bio-based chemicals. Beyond the expense of the antibiotic, its presence in spent fermentation medium poses serious challenges for wastewater treatment systems that rely on biological processes to clean up the water. Furthermore, the antibiotic resistance gene itself may be undesirable at industrial scales: because of public perception and the fears of horizontal gene transfer and the spread of antibiotic resistance into wild populations of bacteria, the presence of antibiotic resistance genes in industrial strains of bacteria is best avoided. Likewise, in medical biotechnology, antibiotics and antibiotic resistance genes are undesirable. For example, allergies to antibiotics are quite common; therefore, commercial processes for the production of therapeutic proteins avoid the use of antibiotics.

Several methods of selecting for plasmids without antibiotics have been developed and practiced in commercial settings (reviewed in: Kroll, J., Klinter, S., Schneider, C., Voβ, I. and A. Steinbüchel. 2010. Plasmid addiction systems: perspectives and applications in biotechnology. Microbiol. Biotechnol. 3: 634-657). Broadly speaking, these methods fall into the categories of toxin/antitoxin systems, metabolism-based systems, and operator-repressor titration systems. Metabolism-based systems are based on the absolute necessity of a key enzyme for cell viability: plasmid DNA encoding the key enzyme is introduced into a host cell where the chromosomal copy of the gene encoding the enzyme is deleted, disrupted or mutated. For a cell to remain viable, it must retain the plasmid. Metabolism-based systems are extensively used in Saccharomyces cerevisiae where defined medium lacking a required metabolite, often an amino acid, is used to maintain the presence of the plasmid (Strausberg, R. L. and S. L Strausberg. 2001. Overview of protein expression in Saccharomyces cerevisiae. Curr. Protoc. Prot. Sci. 5: 5.6.1-5.6.7). A Ralstonia eutropha H16 mutant strain has been similarly engineered to require a plasmid-borne copy of the eda gene (encoding 2-keto-3-deoxy-6-phosphogluconate aldolase) when grown on either gluconate or fructose as a sole carbon source (Gottschalk, G., Eberhardt, U., and H. G. Schlegel. 1964. Verwertung von Fructose durch Hydrogenomonas H16 (I.). Arch. Mikrobiol. 48: 95-108; Blackkolb, F. and H. G. Schlegel. 1968. Katabolische Repression and Enzymhemmung durch molekularen Wasserstoff bei Hydrogenomonas. Arch. Mikrobiol. 62: 129-143). Generally speaking, plasmids maintained through metabolism-based strategies contain antibiotic resistance genes for ease of plasmid manipulation in the laboratory.

Glycerol is gaining prominence as a useful carbon source for large-scale fermentations. While glycerol may be produced from petrochemical feedstocks, it is also produced as a co-product of the oleochemical and biodiesel industries, generally through acid splitting or transesterification of oils and fats from animal or vegetable origin. With the growth of the oleochemical and biodiesel industries, there is now an abundance of glycerol available for use as a carbon source in fermentations.

While examples of metabolism-based plasmid maintenance are common in the literature, the use of glycerol as a sole carbon source to maintain a plasmid in the absence of an antibiotic, and more particularly, in the absence of an antibiotic resistance gene encoded on the plasmid, has not been reported. It would be desirable to use low-cost glycerol as a carbon source in medical and industrial biotechnology, without resorting to the use of antibiotics or antibiotic resistance genes, for the production of biofuels, biochemicals, industrial enzymes, therapeutic proteins, or therapeutic plasmid DNA. However, because of the complex and redundant systems for glycerol dissimilation in many microorganisms, it was not clear that a glycerol-based plasmid maintenance system could be engineered.

### SUMMARY OF THE INVENTION

Embodiments of the present invention generally provide isolated transformed microbial host cells comprising a deletion, a disruption or a mutation that reduces or eliminates the activity of one or more chromosomal genes encoding one or more enzymes of a glycerol dissimilation pathway and a plasmid encoding one or more genes of a glycerol dissimilation pathway operably linked to a promoter, wherein the plasmid lacks an antibiotic resistance gene and the plasmid is stably maintained in the isolated transformed microbial host cell when grown in the presence of glycerol. Preferably, in all embodiments provided herein, the glycerol is the sole or substantially the sole carbon source provided to the host cell.

The host cell may be selected from the genus Escherichia, Salmonella, Enterobacter, Klebsiella, Citrobacter, and Bacillus.

In one preferred embodiment, the chromosomal gene encoding glycerol dehydrogenase is deleted, disrupted or mutated and the plasmid encodes a glycerol dehydrogenase or a glycerol kinase. In another preferred embodiment, the chromosomal gene encoding glycerol kinase is deleted, disrupted or mutated and the plasmid encodes a glycerol kinase or a glycerol dehydrogenase. In another embodiment, the chromosomal gene encoding glycerol dehydrogenase is deleted, disrupted, or mutated, the chromosomal gene encoding glycerol kinase is deleted, disrupted, or mutated, and the plasmid encodes a glycerol kinase. In another embodiment, the chromosomal gene encoding glycerol dehydrogenase is deleted, disrupted, or mutated, the chromosomal gene encoding glycerol kinase is deleted, disrupted, or mutated, and the plasmid encodes a glycerol dehydrogenase. One embodiment of the present invention provides for a microbial host cell comprising a deletion, disruption or mutation of the chromosomal gene encoding glycerol dehydrogenase; a deletion, disruption or mutation of the chromosomal gene encoding glycerol kinase; and the plasmid encodes the gene encoding glycerol kinase and the gene encoding glycerol dehydrogenase.

In an alternative, embodiment, the isolated transformed microbial host cell comprises a deletion, disruption, or mutation in two or more chromosomal genes encoding two or more enzymes of the glycerol dissimilation pathway and two or more plasmids, wherein each plasmid encodes one or more enzymes of the glycerol dissimilation pathway operably linked to a promoter, said plasmids lacking an antibiotic resistance gene. The two or more plasmids are stably maintained in the isolated transformed microbial host cell when grown in the presence of glycerol. The gene of the glycerol dissimilation pathway encoded by the first plasmid may be a glycerol kinase gene and the gene of the glycerol dissimilation pathway encoded by the second plasmid may be a glycerol-3-phosphate dehydrogenase gene, wherein the chromosomal glycerol kinase gene and the chromosomal glycerol-3-phosphate gene are deleted, disrupted, or mutated to reduce or eliminate their activity. Alternatively, the gene of the glycerol dissimilation pathway encoded by the first plasmid may be a glycerol dehydrogenase gene and the gene of the glycerol dissimilation pathway encoded by the second plasmid may be one or more subunits of a dihydroxyacetone kinase operon, wherein the chromosomal glycerol dehydrogenase gene and the one or more subunits of the chromosomal dihydroxyacetone kinase operon are deleted, disrupted, or mutated to reduce or eliminate their activity.

In certain embodiments, a method of maintaining two or more plasmids in a transformed microbial host cell is provided, and the method includes the step of culturing an isolated transformed microbial host cell in the presence of glycerol under conditions sufficient to permit said cell to grow. The host cell comprises a deletion, disruption, or mutation that reduces or eliminates the activity in two or more chromosomal genes encoding two or more enzymes of the glycerol dissimilation pathway and two or more plasmids, wherein each plasmid encodes one or more enzymes of the glycerol dissimilation pathway operably linked to a promoter, said plasmids lacking an antibiotic resistance gene, and wherein the two or more plasmids are stably maintained in the host cell when grown in the presence of glycerol. In one embodiment, the growth conditions are anaerobic or microaerobic and the isolated transformed microbial host cell comprises a deletion, disruption or mutation of the glycerol dehydrogenase gene and one or more subunits of the dihydroxyacetone kinase operon to reduce or eliminate the activities of these enzymes; a first plasmid comprising a glycerol dehydrogenase gene operably linked to a promoter; and, a second plasmid comprising one or more genes encoding subunits of dihydroxyacetone kinase operably linked to a promoter. In another embodiment, the growth conditions are aerobic or microaerobic and the isolated transformed host cell comprises a deletion, disruption or mutation of the glycerol kinase gene and the glycerol-3-phosphate dehydrogenase gene to reduce or eliminate activities of these enzymes; a first plasmid comprising a glycerol kinase gene operably linked to a promoter; and, a second plasmid comprising a glycerol-3-phosphate dehydrogenase gene operably linked to a promoter.

An additional embodiment of the present invention provides for a method of maintaining or stabilizing a plasmid in a transformed microbial host cell in the absence of antibiotics or antibiotic resistance genes by deleting, disrupting or mutating one or more genes of the glycerol dissimilation pathway and including one or more genes of the glycerol dissimilation pathway, operably linked to a promoter, on the plasmid to be stably maintained. The cell is cultured in the presence of glycerol under conditions sufficient to permit said cell to grow. The conditions may be anaerobic and the isolated transformed microbial host cell comprises a deletion, disruption or mutation of the chromosomal gene encoding glycerol dehydrogenase to reduce or eliminate its activity and the plasmid comprises a glycerol dehydrogenase gene operably linked to a promoter. The conditions may be anaerobic and the isolated transformed microbial host cell comprises a deletion, disruption or mutation of the chromosomal gene encoding glycerol dehydrogenase to reduce or eliminate its activity, a deletion, disruption or mutation of the chromosomal gene encoding glycerol kinase to reduce or eliminate its activity, and the plasmid comprises a glycerol dehydrogenase gene operably linked to a promoter. The conditions may be microaerobic and the isolated transformed microbial host cell comprises a deletion, disruption or mutation of the chromosomal gene encoding a glycerol dehydrogenase gene to reduce or eliminate its activity and the plasmid comprises a glycerol dehydrogenase gene operably linked to a promoter. The conditions may be microaerobic and the isolated transformed microbial host cell comprises a deletion, disruption or mutation of the chromosomal gene encoding glycerol dehydrogenase to reduce or eliminate its activity, a deletion, disruption or mutation of the chromosomal gene encoding glycerol kinase to reduce or eliminate its activity, and the plasmid comprises a glycerol dehydrogenase gene operably linked to a promoter. The conditions may be aerobic and the isolated transformed microbial host cell comprises a deletion, disruption or mutation of the chromosomal gene encoding glycerol kinase to reduce or eliminate its activity, and the plasmid comprises a glycerol kinase gene operably linked to a promoter.

The present invention further provides for a method of producing ethanol or other valuable chemicals from glycerol via fermentation, wherein the glycerol is converted to ethanol or another valuable chemical by an isolated transformed microbial host cell comprising: a deletion, disruption or mutation of genes encoding glycerol dehydrogenase and/or glycerol kinase; and, a plasmid comprising at least one of glycerol dehydrogenase and glycerol kinase but lacking an antibiotic resistance gene. In one embodiment, a method of producing ethanol from glycerol comprises culturing an isolated transformed microbial host cell comprising a deletion, disruption, or mutation of one or more chromosomal genes encoding one or more enzymes of the glycerol dissimilation pathway and a plasmid comprising at least one of the one or more genes encoding one or more enzymes of the glycerol dissimilation pathway operably linked to a promoter, said plasmid lacking an antibiotic resistance gene. The cell is cultured in a culture medium containing glycerol under conditions sufficient to permit the cell to grow and produce ethanol. The ethanol is recovered. The host cell may be an E. coli strain that comprises deletions, disruptions, or mutations of the E. coli ldhA, frdA, ackA and/or pta genes to reduce or eliminate their activities. Such modifications may reduce the formation of unwanted by-products such as lactate, succinate, or acetate, and improve the yield of ethanol. The plasmid may encode: both glycerol dehydrogenase and dihydroxyacetone kinase genes operably linked to a promoter; both glycerol kinase and glycerol-3-phosphate dehydrogenase genes operably linked to a promoter; or glycerol kinase, glycerol dehydrogenase, and dihydroxyacetone kinase genes operably linked to a promoter.

Embodiments of the invention further provide a method of producing plasmid DNA comprising culturing an isolated transformed microbial host cell in the presence of glycerol under conditions sufficient to permit the cell to grow and isolating the plasmid DNA from the cell. The cell comprises a deletion, a disruption or a mutation that reduces or eliminates the activity of one or more chromosomal genes encoding one or more enzymes of a glycerol dissimilation pathway and a plasmid encoding one or more genes of a glycerol dissimilation pathway operably linked to a promoter, wherein the plasmid lacks an antibiotic resistance gene and the plasmid is stably maintained in the isolated transformed microbial host cell when grown in the presence of glycerol. The plasmid further comprises a gene or sequence of interest. As the cell is grown in the absence of antibiotics and an antibiotic resistance gene, the plasmid DNA including the gene or sequence of interest should be substantially free of antibiotics, which is desirable for therapeutic use, such as in the context of DNA vaccines or gene therapy.

Embodiments of the invention further provide a method of producing recombinant protein comprising culturing an isolated transformed microbial host cell in the presence of glycerol under conditions sufficient to permit the cell to grow and isolating the recombinant protein. The cell comprises a deletion, a disruption or a mutation that reduces or eliminates the activity of one or more chromosomal genes encoding one or more enzymes of a glycerol dissimilation pathway and a plasmid encoding one or more genes of a glycerol dissimilation pathway operably linked to a promoter, wherein the plasmid lacks an antibiotic resistance gene and the plasmid is stably maintained in the isolated transformed microbial host cell when grown in the presence of glycerol. The plasmid also encodes a recombinant protein of interest. As the cell is grown in the absence of antibiotics and an antibiotic resistance gene, the recombinant protein should be substantially free of antibiotics, which is desirable for therapeutic use.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows: A. A glycerol dissimilation pathway that uses glycerol kinase and either an aerobic glycerol-3-phosphate dehydrogenase (encoded by glpD in Escherichia coli) or an anaerobic glycerol-3-phosphate dehydrogenase (encoded by glpABC in Escherichia coli); and B. A glycerol dissimilation pathway that uses glycerol dehydrogenase and either a phosphoenolpyruvate-dependent dihydroxyacetone kinase or an adenosine triphosphate-(ATP-) dependent dihydroxyacetone kinase.

FIG. 2 shows the growth of strains derived from Escherichia coli ATCC 8739 (GEM6, GEM6, GEM7 GEM8, GEM5(pGB1098), GEM8(pGB1132), GEM5(pGB1166), and GEM8(pGB1133)) on a minimal medium containing glycerol as a sole carbon source under aerobic conditions.

FIG. 3 shows frequencies of plasmid retention for GEM5(pGB1098), GEM8(pGB1132), GEM5(pGB1166) and GEM8(pGB1133) after 24 hours of growth on a minimal medium containing glycerol as a sole carbon source under aerobic conditions.

FIG. 4 shows the growth of strains derived from Escherichia coli ATCC 8739 (GEM5, GEM6, GEM7 GEM8, GEM5(pGB1098), GEM8(pGB1132), GEM5(pGB1166), and GEM8(pGB1133)) on a minimal medium containing glycerol as a sole carbon source under microaerobic conditions.

FIG. 5 shows frequencies of plasmid retention for GEM5(pGB1098), GEM8(pGB1132), GEM5(pGB1166) and GEM8(pGB1133) after 24 hours of growth on a minimal medium containing glycerol as a sole carbon source under microaerobic conditions.

FIG. 6 shows the growth of strains derived from Escherichia coli ATCC 8739 (GEM5, GEM6, GEM7 GEM8, GEM5(pGB1098), GEM8(pGB1132), GEM5(pGB1166), and GEM8(pGB1133)) on a minimal medium containing glycerol as a sole carbon source under anaerobic conditions.

FIG. 7 shows frequencies of plasmid retention for GEM5(pGB1098) and GEM8(pGB1132), after 48 hours of growth on a minimal medium containing glycerol as a sole carbon source under anaerobic conditions.

FIG. 8 shows the growth of GEM5(pGB1098), with and without chloramphenicol, and GEM8(pGB1132) on a minimal medium containing glycerol as a sole carbon source under microaerobic conditions (+CM, with chloramphenicol supplementation; −CM, without chloramphenicol supplementation).

FIG. 9 shows glycerol consumption of GEM5(pGB1098), with and without chloramphenicol, and GEM8(pGB1132) on a minimal medium containing glycerol as a sole carbon source under microaerobic conditions (+CM, with chloramphenicol supplementation; −CM, without chloramphenicol supplementation).

FIG. 10 shows ethanol production of GEM5(pGB1098), with and without chloramphenicol, and GEM8(pGB1132) on a minimal medium containing glycerol as a sole carbon source under microaerobic conditions (+CM, with chloramphenicol supplementation; −CM, without chloramphenicol supplementation).

FIG. 11 shows plasmid retention after 72 hours of growth of GEM5(pGB1098), with and without chloramphenicol, and GEM8(pGB1132) on a minimal medium containing glycerol as a sole carbon source under microaerobic conditions (+CM, with chloramphenicol supplementation; −CM, without chloramphenicol supplementation).

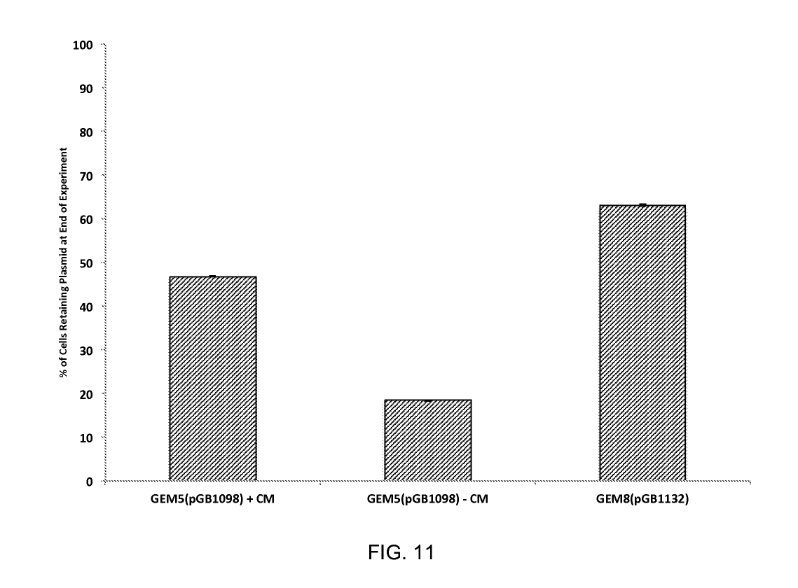

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As defined herein, a knocked-out gene is a gene whose encoded product, e.g., a protein, does not or substantially does not perform its usual function or any function. A knocked-out gene can be created through deletion, disruption, insertion, or mutation. As defined herein, microorganisms that lack one or more indicated knocked-out genes are also considered to have knock outs of the indicated gene(s). The microorganisms themselves may also be referred to as knock outs of the indicated gene(s). Such knock outs can also be conditional or inducible, using techniques that are well-known to those of skill in the art. Also contemplated are “knock ins”, in which a gene, or one or more segments of a gene, are introduced into the microorganism in place of, or in addition to, the endogenous copy of the gene. Once again, many techniques for creating knock in microorganisms are known to those of ordinary skill in the art.

The methods and techniques utilized for culturing or generating the microorganisms disclosed herein are known to the skilled worker trained in microbiological and recombinant DNA techniques. Methods and techniques for growing microorganisms (e.g., bacterial cells), transporting isolated DNA molecules into the host cell and isolating, cloning and sequencing isolated nucleic acid molecules, knocking out expression of specific genes, etc., are examples of such techniques and methods. These methods are described in many items of the standard literature, which are incorporated herein in their entirety: “Basic Methods In Molecular Biology” (Davis, et al., eds. McGraw-Hill Professional, Columbus, Ohio, 1986); Miller, “Experiments in Molecular Genetics” (Cold Spring Harbor Laboratory Press, Cold Spring Harbor, N.Y., 1972); Miller, “A Short Course in Bacterial Genetics” (Cold Spring Harbor Laboratory Press, Cold Spring Harbor, N.Y., 1992); Singer and Berg, “Genes and Genomes” (University Science Books, Mill Valley, Calif., 1991); “Molecular Cloning: A Laboratory Manual,” 2^{nd} Ed. (Sambrook, et al., eds., Cold Spring Harbor Laboratory Press, Cold Spring Harbor, N.Y., 1989); “Handbook of Molecular and Cellular Methods in Biology and Medicine” (Kaufman, et al., eds., CRC Press, Boca Raton, Fla., 1995); “Methods in Plant Molecular Biology and Biotechnology” (Glick and Thompson, eds., CRC Press, Boca Raton, Fla., 1993); and Smith-Keary, “Molecular Genetics of Escherichia coli” (The Guilford Press, New York, N.Y., 1989).

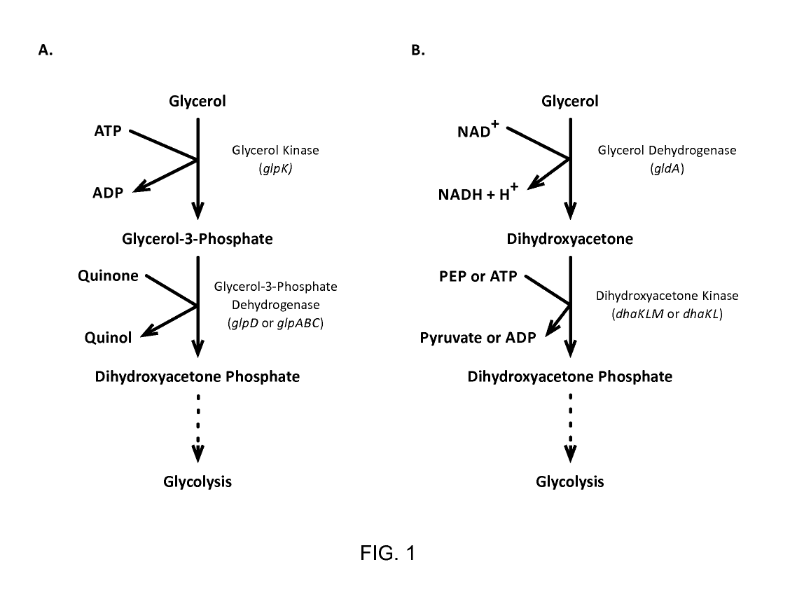

Two primary metabolic pathways account for the major routes of glycerol dissimilation in bacteria (FIG. 1). One pathway uses glycerol kinase to convert glycerol to glycerol-3-phosphate (G3P) and glycerol-3-phosphate dehydrogenase to convert G3P to the key metabolite dihydroxyacetone phosphate (DHAP). A second pathway uses glycerol dehydrogenase to convert glycerol to dihydroxyacetone (DHA) and dihydroxyacetone kinase to convert DHA to DHAP. Under aerobic or microaerobic conditions, glycerol kinase is essential for growth on glycerol. A deletion of glpK, the gene encoding glycerol kinase.

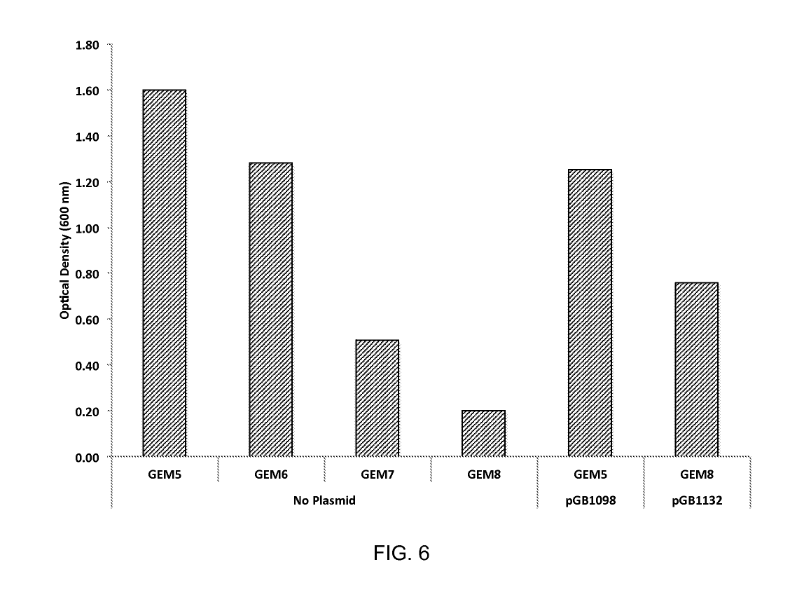

Table 4), abolishes the ability to grow aerobically (compare the growth of GEM5 and GEM6), or microaerobically (compare the growth of GEM5 and GEM6), on glycerol as a sole carbon source. Under anaerobic conditions, glycerol dehydrogenase is essential for growth on glycerol. A deletion of gldA, the gene encoding glycerol dehydrogenase, abolishes the ability to grow anaerobically (Gonzalez, R., Murarka, A., Dharmadi, Y. and S. S. Yazdani. 2008. A new model for the anaerobic fermentation of glycerol in enteric bacteria: trunk and auxiliary pathways in Escherichia coli. Metabol. Eng. 10: 234-245; and, compare the growth of GEM5 and GEM6, FIG. 6).

The present invention provides strains and plasmids that, when used together, stably maintain the plasmid in a growing population of cells without the use of antibiotics or antibiotic-selectable markers. Host strains lacking genes involved in glycerol dissimilation are unable to grow on glycerol as a sole carbon source. Genes permitting glycerol dissimilation are provided in trans, that is, on a plasmid DNA molecule. As defined herein, plasmids include cosmids, bacterial artificial chromosomes (BACs) and other non-chromosomal DNA elements, both linear and circular forms, present in the cell. Preferably, the plasmid lacks an antibiotic selectable gene or marker.

### Strains

The common E. coli cloning strain DH5α, TRANSFORMAX EC100D pir¹ , and strain ECK3918 were used during construction of all vectors. For testing the plasmid constructs, ATCC 8739 or its derivatives were used as the host strain. Vectors that were constructed in house were transformed into chemically competent DH5αcells (also referred to as GC5) or electrocompetent TRANSFORMAX EC100D pir⁺cells. Cells were made electrocompetent and electroporated following the protocol from the MICROPULSER Electroporation Apparatus Operating Instructions and Applications Guide (Bio-Rad catalog number 165-2100), except that Luria Bertani broth (5 grams/liter yeast extract, 10 grams/liter tryptone) without salt was used to grow up the culture in making cells electrocompetent. Strain genotypes are found in Table 1. Plasmids that were used are listed in Table 2, and primers are listed in Table 3. Genes are listed in Table 4.

TABLE 1
STRAINS

| Strain | Genotype | Reference |
|---|---|---|
| ATCC 8739 | Wild type E. coli | ATCC* 8739 |
| GC5 (DH5α) | E. coli (F⁻φ80lacZΔM15 Δ(lacZYA-argF)U169 | Sigma-Aldrich Co. LLC, |
| | recA1 endA1 hsdR17(rk⁻, mk⁺) phoA supE44 | www.sigmaaldrich.com |
| | thi-1 gyrA96 relA1 λ⁻ tonA) | |
| TRANSFORMAX ™ | F⁻ mcrA Δ(mrr-hsdRMS-mcrBC) | Epicentre Biotechnologies |
| EC100D ™ pir⁺ | φ80dlacZΔM15 ΔlacX74 recA1 endA1 araD139 | (Illumina, Inc.; |
| | Δ(ara, leu)7697 galU galK λ⁻ rpsL (Str^{R}) nupG | www.epibio.com) |
| | pir⁺ (DHFR) | |
| ECK3918 | E. coli BW25113 (F⁻ Δ(araD-araB)567 | Keio Collection (Baba, T., Ara, |
| | ΔlacZ4787(::rrnB-3) ⁻rph-1 Δ(rhaD- | T., Hasegawa, M., Takai, Y., |
| | rhaB)568 hsdR514) glpK::Km^{R} | Okumura, Y., Baba, M., |
| | | Datsenko, K. A., Tomita, M., |
| | | Wanner, B. L., and H. Mori. |
| | | 2006. Construction of |
| | | Escherichia coli K-12 in-frame, |
| | | single-gene knockout mutants: |
| | | the Keio collection. Mol. Syst. |
| | | Biol. 2: 2006 0008 |
| | | 10.1038/msb4100050). |
| GEM2 | ATCC 8739 ΔldhA_1Kb | Glycos Biotechnologies, Inc. |
| GEM3 | GEM2 ΔackA/pta | Glycos Biotechnologies, Inc. |
| GEM4 | GEM2 ΔackA/pta ΔpoxB | Glycos Biotechnologies, Inc. |
| GEM5 | GEM2 ΔackA/pta ΔpoxB ΔfrdA | Glycos Biotechnologies, Inc. |
| GEM6 | GEM2 ΔackA/pta ΔpoxB ΔfrdA ΔglpK | Glycos Biotechnologies, Inc. |
| GEM7 | GEM2 ΔackA/pta ΔpoxB ΔfrdA ΔgldA | Glycos Biotechnologies, Inc. |
| GEM8 | GEM2 ΔackA/pta ΔpoxB ΔfrdA ΔglpK ΔgldA | Glycos Biotechnologies, Inc. |

*American Type Culture Collection

TABLE 2
PLASMIDS

| Plasmid | Features | Source |
|---|---|---|
| pR6Kan | pGB704 containing the tn903 kanamycin | Orchard, S. S., and H. Goodrich- |
| | resistance marker | Blair. 2005. Pyrimidine |
| | | nucleoside salvage confers an |
| | | advantage to Xenorhabdus |
| | | nematophila in its host |
| | | interactions. Appl. Env. |
| | | Microbiol. 71: 6254-6259. |
| pR6KT-intermediate | pR6Kan fragment containing upstream and | Glycos Biotechnologies, Inc. |
| | downstream portion of the E. coli ldhA locus | |
| pR6KT-ΔldhA | pR6KT-intermediate containing the tetRA | Glycos Biotechnologies, Inc. |
| | region from transposon Tn10; for deleting ldhA | |
| | in E. coli | |
| pR6KT-ΔackApta | pR6KT-ΔldhA derivative for deleting ackA/pta | Glycos Biotechnologies, Inc. |
| | in E. coli | |
| pR6KT-ΔpoxB | pR6KT-ΔldhA derivative for deleting poxB in | Glycos Biotechnologies, Inc. |
| | E. coli | |
| pR6KT-ΔldhA_1Kb | pR6KT-ΔldhA derivative for deleting ldhA in | Glycos Biotechnologies, Inc. |
| | E. coli | |
| pR6KT-ΔfrdA | pR6KT-ΔldhA derivative for deleting frdA in | Glycos Biotechnologies, Inc. |
| | E. coli | |
| pR6KT-ΔglpK | pR6KT-ΔldhA derivative for deleting glpK in | Glycos Biotechnologies, Inc. |
| | E. coli | |
| pR6KT-ΔgldA | pR6KT-ΔldhA derivative for deleting gldA in | Glycos Biotechnologies, Inc. |
| | E. coli | |
| pZSKLMGldA | Low-copy expression vector supplying gldA | Yazdani, S. S., and R. Gonzalez. |
| | and dhaKLM in trans | 2008. Engineering Escherichia |
| | | coli for the efficient conversion |
| | | of glycerol to ethanol and co- |
| | | products. Metabol. Eng. 10: |
| | | 340-351. |
| pGB1165 | pZSKLMGldA derivative, PLtetO-1 is replaced | |
| | with the adhE promoter region | |
| pGB1098 | pGB1165 derivative; adding rbs site in front of | Glycos Biotechnologies, Inc. |
| | gldA, chloramphenicol resistance | |
| pGB1132 | pGB1098 derivative; chloramphenicol marker | Glycos Biotechnologies, Inc. |
| | deleted | |
| pZS.glpK.glpD | Low-copy expression vector supplying glpK | Durnin et al., 2009 |
| | and glpD in trans, chloramphenicol resistance, | |
| | anhydrotetracycline-inducible | |
| pGB1166 | pZS.glpK.glpD derivative, PLtetO-1 is | Glycos Biotechnologies, Inc. |
| | replaced with the adhE promoter region | |
| pGB1133 | Derivative of pGB1166; Cm marker deleted | Glycos Biotechnologies, Inc. |

TABLE 3

| PRIMERS | |
|---|---|
| Primer Name | Primer Sequence (5′ to 3′) |
| P1 | ATC GAG CTC AAC CAT CAT CGA TGA ATT GC |
| P2 | TCT GGT ACC CTG AAG CTT GCA TGC C |
| P3 | ATA GAG CTC CCA CCA GAT AAC GG |
| P4 | GCG CTC GAG TTT CAT AAG ACT TTC TCC |
| P5 | GAC CTC GAG CTG GTT TAA TCT TGC CG |
| P6 | CGT GGT ACC TTC TTT CAG CAT TTC G |
| P7 | ACC GTG GCG GGG ATC TTA AGA CCC ACT TTC ACA TTT AAG TTG T |
| P8 | CGA CTC TAG AGG ATC CTA AGC ACT TGT CTC CTG TTT ACT C |
| P9 | ACG GAG CTC ATC TTG ATA ACG CGA T |
| P10 | TAC CTC GAG CAT GGA AGT ACC TAT AAT TG |
| P11 | GGA TAA ACC GTG TCC CTC GAG CAG CAG TAA TCT CG |
| P12 | ACT GGT ACC TCT AGA AGT TCA TCA GGC C |
| P13 | TGA GAG CTC AGA GCA GGA AGT GAA AGC |
| P14 | CCT TTT TAC CTT AGC CAG TTT GTA CAT TTC ATG GTT CTC CAT CTC C |
| P15 | ACA AAC TGG CTA AGG TAA AAA GG |
| P16 | AGT GGT ACC GAC TGA GTA ATT TGG TGC G |
| P17 | ATA GAG CTC AAA TCG AGC AAC GGA TCG |
| P18 | CGG CAA GAT TAA ACC AGT TTG TAC AGT TTC ATA AGA CTT TCT CCA G |
| P19 | CTG GAG AAA GTC TTA TGA AAC TGT ACA AAC TGG TTT AAT CTT GCC G |
| P20 | TCA GGT ACC AAT GTT TTG ATC AAA CAG AGG G |
| P21 | ATT GAG CTC GGT TAC CGT CGC C |
| P22 | GCC ATT CGC CTT CTC GAG CAC GAC ATT CCT CC |
| P23 | GGA GGA ATC TCG TGC TCG AGA AGG CGA ATG GC |
| P24 | AGC GGT ACC ACA GTT GAT GCA ACC |
| P25 | AAA AAA GGT ACC GCG TTG AAA GTG TTG ATC TG |
| P26 | TTA TTC GTC GTG TTC GTT TAA TTG TCC CGT AGT CAT ATT AC |
| P27 | GAA CAC GAC GAA TAA CTC GAG TGT AAA TGC CGA ATC AAG CG |
| P28 | AAA AAA GAG CTC CAG ATC AAT GGT GCC TTT GG |
| P29 | TTT GGT ACC GCA TTC TGT TTG CTC AGA CTA TG |
| P30 | TTA TTC CCA CTC TTG AAT TGC TCC TTT AGA GAT GAG TAG TG |
| P31 | CAA GAG TGG GAA TAA CTC GAG CCT ACT CCA AAC TCC CGG |
| P32 | TTT TTT GAG CTC GTT TCT GCA AAT CGA CCT TCT G |
| P33 | CGC CTC GAG GGT TAG CTC CGA AGC AAA AGC |
| P34 | CGC GGT ACC AAT GCT CTC CTG ATA ATG TTA AAC |
| P35 | ACC GTC AGG GTT AAC TGC AGA AAT AGG AGA TAA AAT ATA TGG ACC |
| | GCA TTA TTC AAT CAC CGC |
| P36 | TGC CTC TAG CAC GCG TTT ATT CCC ACT CTT GCA GGA AAC GC |
| P37 | CGC CTC GAG GGT TAG CTC CGA AGC AAA AGC |
| P38 | CGC GGT ACC AAT GCT CTC CTG ATA ATG TTA AAC |

TABLE 2 Note:
restriction sites used in plasmid construction are underlined.

TABLE 4
GENE ABBREVIATIONS

| Name | Gene(s) | Organism | Ecocyc Reference |
|---|---|---|---|
| Phosphate acetyltransferase | pta | E. coli | EG20173 |
| Acetate kinase | ackA | E. coli | EG10027 |
| Pyruvate oxidase | poxB | E. coli | EG10754 |
| Lactate dehydrogenase | ldhA | E. coli | G592 |
| Fumarate reductase subunit | frdA | E. coli | EG10330 |
| Glycerol kinase | glpK | E. coli | EG10398 |
| Glycerol dehydrogenase | gldA | E. coli | EG11904 |

### Cloning Methods and Plasmids

To create the basic suicide vector used to delete genes from the E. coli chromosome, the R6Ky origin of replication, kanamycin marker and multiple cloning site of plasmid pR6Kan (Orchard, S. S., and H. Goodrich-Blair. 2005. Pyrimidine nucleoside salvage confers an advantage to Xenorhabdus nematophila in its host interactions. Appl. Environ. Microbiol. 71:6254-6259) was amplified by polymerase chain reaction (PCR) using primers P1 and P2, which introduce SacI and KpnI restriction enzyme sites, respectively. An approximately 500 base pair (bp) fragment upstream of the E. coli ldhA gene was PCR amplified using primers P3 and P4, which introduce SacI and XhoI restriction sites, respectively. An approximately 500 base pair fragment downstream of the E. coli ldhA gene was PCR amplified using primers P5 and P6, which introduce XhoI and SacI restriction sites, respectively. The three PCR products were restriction-digested with SacI, XhoI and/or KpnI restriction enzymes as appropriate, and the resulting fragments were used in a trimolecular ligation reaction with the QUICK LIGASE KIT (New England Biolabs, Ipswich, Mass.). Restriction enzymes and buffer components were removed from the ligation reaction using the DNA CLEAN AND CONCENTRATOR KIT (Zymo Research, Irvine, Calif.) before electroporation into TRANSFORMAX EC100D pir+ electrocompetent cells (Epicentre Biotechnologies, Madison, Wis.). Transformants were selected on LB plates containing 50 μg/ml kanamycin and the correct plasmid identified by restriction digestion. The resulting plasmid, pR6KT-intermediate, was then further modified by introduction of the tetRA locus as a counterselectable marker as follows: the intermediate plasmid was restriction digested using BamHI, while the tetRA locus was PCR amplified using primers P7 and P8; the two DNA fragments were joined using the IN-FUSION CLONING SYSTEM (Clontech, Mountain View, Calif.), diluted five-fold with water, then electroporated into TRANSFORMAX EC100D pir+ electrocompetent cells. Transformants were selected on LB plates containing 50 μg/ml kanamycin and the correct plasmid identified by restriction digestion. This plasmid is named pR6KT-ΔldhA.

pR6KT-ΔackApta. Plasmid pR6KT-ΔackApta was constructed as follows. An approximately 462 base pair fragment upstream of the E. coli ackA gene was PCR amplified using primers P9 and P10, which introduce SacI and XhoI restriction enzyme sites, respectively. The resulting PCR product was restriction digested with SacI and XhoI. An approximately 473 base pair fragment downstream of the E. coli pta gene was PCR amplified using primers P11 and P12, which introduce XhoI and KpnI restriction enzyme sites, respectively. The resulting PCR product was restriction digested with XhoI and KpnI. Plasmid pR6KT-ΔldhA was restriction digested with SacI and KpnI followed by purification by agarose gel electrophoresis. The band corresponding to the vector backbone was recovered from the agarose using the GEL DNA RECOVERY KIT (Zymo Research). The three restriction-digested DNA fragments corresponding to the vector backbone, the upstream region of ackA, and the downstream region of pta were used in a trimolecular ligation reaction with the QUICK LIGASE KIT (New England Biolabs, Ipswich, Mass.). Restriction enzymes and buffer components were removed from the ligation reaction using the DNA CLEAN AND CONCENTRATOR KIT (Zymo Research) before electroporation into TRANSFORMAX EC100D pir+ electrocompetent cells (Epicentre Biotechnologies). Transformants were selected on LB plates containing 50 μml/kanamycin and the correct plasmid identified by restriction digestion.

pR6KT-ΔpoxB. Plasmid pR6KT-ΔpoxB was constructed as follows. An approximately 458 base pair fragment upstream of the E. coli poxB gene was PCR amplified using primers P13 and P14, which introduce SacI and BsrGI restriction enzyme sites, respectively. An approximately 454 base pair fragment downstream of the E. coli poxB gene was PCR amplified using primers P15 and P16, which introduce a KpnI restriction enzyme site. The two resulting PCR products were joined using overlap extension PCR (OE-PCR) with primers P13 and P16. The resulting PCR product was digested with SacI and KpnI. Plasmid pR6KT-ΔldhA was restriction digested with SacI and KpnI followed by purification by agarose gel electrophoresis. The band corresponding to the vector backbone was recovered from the agarose using the GEL DNA RECOVERY KIT (Zymo Research). The two restriction-digested DNA fragments corresponding to the vector backbone, and the fused upstream and downstream regions of poxB were used in a ligation reaction with the QUICK LIGASE KIT (New England Biolabs, Ipswich, Mass.). Restriction enzymes and buffer components were removed from the ligation reaction using the DNA CLEAN AND CONCENTRATOR KIT (Zymo Research) before electroporation into TRANSFORMAX EC100 pir+ electrocompetent cells (Epicentre Biotechnologies). Transformants were selected on LB plates containing 50 μg/ml kanamycin and the correct plasmid identified by restriction digestion.

pR6KT-ΔldhA₁₃ 1Kb. Plasmid pR6KT-ΔldhA_{—}1Kb was constructed as follows. An approximately 942 base pair fragment upstream of the E. coli ldhA gene was PCR amplified using primers P17 and P18, which introduce SacI and BsrGI restriction enzyme sites, respectively. An approximately 957 base pair fragment downstream of the E. coli ldhA gene was PCR amplified using primers P19 and P20, which introduce BsrGI and KpnI restriction enzyme sites, respectively. The two resulting PCR products were joined using overlap extension PCR (OE-PCR) with primers P17 and P20. The resulting PCR product was digested with SacI and KpnI. Plasmid pR6KT-ΔldhA was restriction digested with SacI and KpnI followed by purification by agarose gel electrophoresis. The band corresponding to the vector backbone was recovered from the agarose using the GEL DNA RECOVERY KIT (Zymo Research). The two restriction-digested DNA fragments corresponding to the vector backbone, and the fused upstream and downstream regions of ldhA were used in a ligation reaction with the QUICK LIGASE KIT (New England Biolabs, Ipswich, Mass.). Restriction enzymes and buffer components were removed from the ligation reaction using the DNA CLEAN AND CONCENTRATOR KIT (Zymo Research) before electroporation into TRANSFORMAX EC100D pir+ electrocompetent cells (Epicentre Biotechnologies). Transformants were selected on LB plates containing 50 μg/ml kanamycin and the correct plasmid identified by restriction digestion.

pR6KT-ΔfrdA. Plasmid pR6KT-ΔfrdA was constructed as follows. An approximately 448 base pair fragment upstream of the E. coli frdA gene was PCR amplified using primers P21 and P22, which introduce SacI and XhoI restriction enzyme sites, respectively. An approximately 471 base pair fragment downstream of the E. coli frdA gene was PCR amplified using primers P23 and P24, which introduce XhoI and KpnI restriction enzyme sites, respectively. The two resulting PCR products were joined using overlap extension PCR (OE-PCR) with primers P21 and P24. The resulting PCR product was digested with SacI and KpnI. Plasmid pR6KT-ΔldhA was restriction digested with SacI and KpnI followed by purification by agarose gel electrophoresis. The band corresponding to the vector backbone was recovered from the agarose using the GEL DNA RECOVERY KIT (Zymo Research). The two restriction-digested DNA fragments corresponding to the vector backbone, and the fused upstream and downstream regions of frdA, were used in a ligation reaction with the QUICK LIGASE KIT (New England Biolabs). Restriction enzymes and buffer components were removed from the ligation reaction using the DNA CLEAN AND CONCENTRATOR KIT (Zymo Research) before electroporation into TRANSFORMAX EC100D pir+ electrocompetent cells (Epicentre Biotechnologies). Transformants were selected on LB plates containing 50 μg/ml kanamycin and the correct plasmid identified by restriction digestion.

pR6KT-ΔglpK. Plasmid pR6KT-ΔglpK was constructed as follows. An approximately 495 base pair fragment upstream of the E. coli glpK gene was PCR amplified using primers P25 and P26, which introduce a KpnI restriction site. An approximately 539 base pair fragment downstream of the E. coli glpK gene was PCR amplified using primers P27 and P28, which introduce XhoI and SacI restriction enzyme sites, respectively. The two resulting PCR products were joined using overlap extension PCR (OE-PCR) with primers P25 and P28. The resulting PCR product was digested with SacI and KpnI. Plasmid pR6KT-ΔldhA was restriction digested with SacI and KpnI followed by purification by agarose gel electrophoresis. The band corresponding to the vector backbone was recovered from the agarose using the GEL DNA RECOVERY KIT (Zymo Research). The two restriction-digested DNA fragments corresponding to the vector backbone, and the fused upstream and downstream regions of glpK were used in a ligation reaction with the QUICK LIGASE KIT (New England Biolabs). Restriction enzymes and buffer components were removed from the ligation reaction using the DNA CLEAN AND CONCENTRATOR KIT (Zymo Research) before electroporation into TRANSFORMAX EC100D pir+ electrocompetent cells (Epicentre Biotechnologies). Transformants were selected on LB plates containing 50 μg/ml kanamycin and the correct plasmid identified by restriction digestion.

pR6KT-ΔgldA. Plasmid pR6KT-ΔgldA was constructed as follows. An approximately 513 base pair fragment upstream of the E. coli gldA gene was PCR amplified using primers P29 and P30, which introduce a KpnI restriction site. An approximately 548 base pair fragment downstream of the E. coli gldA gene was PCR amplified using primers P31 and P32, which introduce XhoI and SacI restriction enzyme sites, respectively. The two resulting PCR products were joined using overlap extension PCR (OE-PCR) with primers P29 and P32. The resulting PCR product was digested with SacI and KpnI. Plasmid pR6KT-ΔldhA was restriction digested with SacI and KpnI followed by purification by agarose gel electrophoresis. The band corresponding to the vector backbone was recovered from the agarose using the GEL DNA RECOVERY KIT (Zymo Research). The two restriction-digested DNA fragments corresponding to the vector backbone, and the fused upstream and downstream regions of gldA were used in a ligation reaction with the QUICK LIGASE KIT (New England Biolabs). Restriction enzymes and buffer components were removed from the ligation reaction using the DNA CLEAN AND CONCENTRATOR KIT (Zymo Research) before electroporation into TRANSFORMAX EC100D pir+ electrocompetent cells (Epicentre Biotechnologies). Transformants were selected on LB plates containing 50 μg/ml kanamycin and the correct plasmid identified by restriction digestion.

pGB1165. Plasmid pGB1165 was constructed by inserting an adhE promoter at the XhoI and KpnI restriction sites of plasmid pZSKLMGldA, replacing the PLteto-1 promoter. Plasmid pZSKLMGldA was first restriction digested with PvuI to isolate, and save for later, a fragment of the plasmid that had secondary KpnI restriction sites. The larger band was purified by agarose gel electrophoresis, and fused together in a ligation reaction using the QUICK LIGASE KIT (NEB). The ligation reaction was purified using the DNA CLEAN AND CONCENTRATOR KIT (Zymo Research) before electroporation into GC5 electrocompetent cells. Transformants were selected on LB plates containing 20 μg/ml chloramphenicol and the correct plasmid identified by restriction digestion. This intermediate plasmid was then restriction digested with XhoI and KpnI, as was a PCR fragment that encoded the adhE promoter that was amplified from MG1655 using ACCUPRIME Pfx polymerase with oligonucleotide primers P33 and P34, incorporating XhoI and KpnI restriction sites, respectively. The PCR product and intermediate plasmid were ligated together with a QUICK LIGASE KIT (New England Biolabs). The ligation reaction was purified using the DNA CLEAN AND CONCENTRATOR KIT (Zymo Research) before electroporation into GC5 competent cells. Transformants were selected on LB plates containing 20 μg/ml chloramphenicol and the correct plasmid identified by restriction digestion. This second intermediate plasmid was finally restriction digested with PvuI and the PvuI/PvuI fragment isolated previously was placed into the vector in a ligation reaction using the QUICK LIGASE KIT (NEB). The ligation reaction was purified using the DNA CLEAN AND CONCENTRATOR KIT before electroporation into GC5 electrocompetent cells. Transformants were selected on LB plates containing 20 μg/ml chloramphenicol and the correct plasmid identified by restriction digestion and functionality.

pGB1098. Plasmid pGB1098 was constructed by inserting a ribosome binding site in front of the gldA coding region at the PstI and MluI restriction sites of pGB1165. The PCR product encoding the ribosome binding site and gldA was amplified from genomic DNA prepared from E. coli MG1655 using PHUSION polymerase with oligonucleotide primers P35 and P36, incorporating PstI and MluI restriction sites, respectively. Both primers included appropriate vector-overlapping 5′ sequences for use with the IN-FUSION ADVANTAGE PCR CLONING KIT. The PCR product was gel-purified, as was pGB1165 linearized with the restriction endonuclease MluI. Fragments were directionally joined together using the In-Fusion cloning kit and GC5 competent cells, following the manufacturer's directions. Transformants were screened, and the proper plasmid was identified through agarose gel electrophoresis of restriction endonuclease-digested plasmid DNAs.

pGB1132. Plasmid pGB1132 was constructed as follows. Plasmid pGB1098 was restriction digested with SacI and AatII, restriction sites flanking the gene encoding chloramphenicol resistance. The larger DNA fragment was purified by agarose gel electrophoresis followed by treatment with a QUICK BLUNTING KIT (New England Biolabs). The blunt-ended vector fragment was re-circularized using a QUICK LIGASE KIT (New England Biolabs). The ligation reaction was purified using the DNA CLEAN AND CONCENTRATOR KIT before electroporation into GEM8 electrocompetent cells. Transformants were positively selected on MM29 minimal medium plates supplemented with 40 g/l glycerol and negatively selected on LB plates containing 20 μg/ml chloramphenicol.

pGB1166. Plasmid pGB1166 was constructed by inserting an adhE promoter at the XhoI and KpnI restriction sites of plasmid pZS.glpK.glpD, replacing the PLteto-1 promoter. The adhE promoter amplified by PCR using genomic DNA of E. coli MG1655 as the template and primers P37 and P38. The resulting PCR product was digested with XhoI and KpnI. Likewise, pZS.glpK.glpD was digested with XhoI and KpnI. The two restriction digested fragments corresponding to the vector backbone and the adhE promoter was purified by agarose gel electrophoresis and fused together in a ligation reaction using the QUICK LIGASE KIT (NEB). The ligation reaction was purified using the DNA CLEAN AND CONCENTRATOR KIT before electroporation into GC5 electrocompetent cells. Transformants were selected on LB plates containing 20 μg/ml chloramphenical and the correct plasmid identified by restriction digestion.

pGB1133. Plasmid pGB1133 was constructed as follows. Plasmid pGB1166 was restriction digested with SacI and AatII, restriction sites flanking the gene encoding chloramphenicol resistance. The larger DNA fragment was purified by agarose gel electrophoresis followed by treatment with a QUICK BLUNTING KIT (New England Biolabs). The blunt-ended vector fragment was re-circularized using a QUICK LIGASE KIT (New England Biolabs). The ligation reaction was purified using the DNA CLEAN AND CONCENTRATOR KIT before electroporation into GEM8 electrocompetent cells. Transformants were positively selected on MM29 minimal medium plates supplemented with 40 g/l glycerol and negatively selected on LB plates containing 20 μg/ml chloramphenicol.

### Strain Construction

Gene deletions in E. coli were made using the approach of Metcalf and colleagues (Metcalf, W. W., Jiang, W., Daniels, L. L., Kim, S. K., Haldiman, A. and B. L. Wanner. 1996. Conditionally replicative and conjugative plasmids carrying lacZa for cloning, mutagenesis, and allele replacement in bacteria. Plasmid 35: 1-13), which is described below. E. coli ATCC 8739 strains with various genes deleted were made using suicide vectors. pR6KT-based gene knock-out plasmids were purified from TRANSFORMAX EC100D pir⁺ using standard plasmid DNA purification techniques and concentrated to approximately 1 μg/ml or greater. Concentrated plasmid DNA was electroporated into the pir⁻ recipient strain (ATCC8739 and its derivatives). Transformants were selected on LB agar plates containing 15 μg/ml tetracycline and 2.5 mM Na₄P₂O₇. After confirming integration by testing for resistance to tetracycline, integration into the correct locus was confirmed by PCR. Transformants with the knock-out plasmid integrated into the proper locus were restreaked onto LB agar plates without antibiotic selection to provide the opportunity for chromosomal rearrangement to resolve the gene duplication. Individual colonies from the LB agar plate were then restreaked onto tetracycline-sensitive-selection agar (TSS) and incubated for two or more days at 30° C. Large colonies from the TSS agar plates were then tested for sensitivity to tetracycline and kanamycin using LB agar plates containing either tetracycline (15 μg/ml) or kanamycin (50 μg/ml ). As resolution of the gene duplication can either generate a gene deletion or recreate the wild-type locus, colonies sensitive to both kanamycin and tetracycline were then tested for the desired gene deletion using PCR. Further confirmation was provided by restriction digest of the PCR product with either XhoI or BsrGI (as appropriate).

TSS agar plates were made as follows. 4.347 g NaH₂PO₄ was mixed with 100 mL distilled water. To this solution, the following chemicals were added: 3 ml of fusaric acid, 2 mg/ml; 2.5 mL ZnCl₂, 20 mM; and 0.5 ml anhydrotetracycline, 5 mg/mL. This buffer solution was sterilized by nanofiltration. 2.5 g tryptone, 2.5 g yeast extract, 5 g sodium chloride, and 7.5 g agar were mixed with 400 mL distilled water and autoclaved. Once the agar solution cooled to approximately 45° C., it was mixed with 100 mL of the buffer solution. This final buffer/agar mixture was then poured into 100 mm-diameter petri plates.

Using the above gene deletion method, the GEM series of E. coli strains were made serially. To create GEM2, ATCC 8739 was transformed with pR6KT-ΔldhA_{—}1Kb, and then cured of the integrated plasmid by restreaking on TSS agar. After verification of the deletion of the ldhA locus, GEM2 was transformed with pR6KT-ΔackApta and then cured of the integrated plasmid by restreaking on TSS agar. The resulting strain was named GEM3. After verification of the deletion of the ackA/pta locus, GEM3 was transformed with pR6KT-ΔpoxB and then cured of the integrated plasmid by restreaking on TSS agar. The resulting strain was named GEM4. After verification of the deletion of the poxB locus, GEM4 was transformed with pR6KT-ΔfrdA and then cured of the integrated plasmid by restreaking on TSS agar. The resulting strain was named GEM5. After verification of the deletion of the frdA locus, GEM5 was transformed with pR6KT-ΔglpK and then cured of the integrated plasmid by restreaking on TSS agar. The resulting strain was named GEM6. Deletion of the glpK locus was verified by PCR and restriction digest of the resulting PCR product. To create GEM7, GEMS was transformed with pR6KT-ΔgldA and then cured of the integrated plasmid by restreaking on TSS agar. The resulting strain was named GEM7. Deletion of the gldA locus was verified by PCR and restriction digest of the resulting PCR product. To create GEM8, GEM6 was transformed with pR6KT-ΔgldA and then cured of the integrated plasmid by restreaking on TSS agar. Deletion of the gldA locus was verified by PCR and restriction digest of the resulting PCR product.

### EXAMPLES

In the examples below, ingredients for the minimal medium were as follows: 0.66 g/L (NH₄)₂SO₄; 1.2 g/L Na₂HPO₄; 3.0 g/L NH₄Cl; 0.25 g/L K₂SO₄; 0.4 g/L MgCl₂.6H₂O; 3.0 mg/L FeSO₄.7H₂O; 70.0 mg/L CaCl₂.2H₂O; 0.173 mg/L Na₂O₃Se; 4.0 μg/L (NH₄)₂MoO₄.4H₂O; 25.0 μg/L H₃BO₃;7.0 μg/L CoCl₂.6H₂O; 3.0 μg/L CuSO₄.5H₂O; 16.0 g/L MnCl₂.4H₂O; 3.0 μg/L ZnSO₄.7H₂O.

All experiments were routinely started from strains stored at −80° C. as glycerol stocks. Strains not bearing plasmids were streaked from the appropriate glycerol stock onto LB-agar plates (LB medium is 10 g/L NaCl, 5 g/L yeast extract, 10 g/L tryptone, supplemented with 15 g/L agar). GEM5 strains bearing plasmids were streaked from the appropriate glycerol stock onto LB-agar plates supplemented with 20 μg/mL chloramphenicol. GEM8 strains bearing plasmids were streaked from the appropriate glycerol stock onto minimal medium plates supplemented with 40 g/L glycerol (as above, supplemented with 15 g/L agar). All plates were incubated at 37° C. for 16 to 24 hours to permit the formation of individual colonies.

### Example 1

### Aerobic Conditions

This working example demonstrates complementation of E. coli mutants deficient in glycerol dissimilation by plasmids bearing glycerol dissimilation genes but lacking antibiotic resistance genes, under aerobic conditions. The following E. coli strains were used in this example: GEM5, GEM6, GEM7, GEM8, GEM5(pGB1166), GEM5(pGB1098), GEM8(pGB1132), and GEM8(pGB1133).

Individual colonies of GEM strains not bearing plasmids were used to inoculate 5 mL of LB medium (10 g/L NaCl, 5 g/L yeast extract, 10 g/L tryptone) in a 14-mL, round-bottom, polypropylene culture tube. Individual colonies of GEM5 or GEM8 strains bearing plasmids were used to inoculate 5 mL of minimal medium supplemented with 20 g/L glycerol and 0.1 M Sorensen's phosphate buffer (39.0 mL 0.2 M NaH₂PO₄, 61 mL 0.2 M Na₂HPO₄, pH 7.0) in a 25-mL Erlenmeyer flask. All cultures of GEM5 strains bearing plasmids were also supplemented with 20 μg/mL chloramphenicol. The 5-mL cultures were incubated for 16 hours at 37° C. in a LAB COMPANION SI-600R incubating shaker set at 175 rpm.

Cultures grown in LB medium were pelleted by low-speed centrifugation and washed by resuspension in minimal medium.

The 16-hour cultures were used to seed 1-mL cultures of minimal medium supplemented with 20 g/L glycerol and 0.1 M Sorensen's phosphate buffer, pH 7.0, in a 24-well deep-well microtiter plate (Enzyscreen, Haarlem, Netherlands). All experimental cultures were seeded with sufficient cells to achieve an initial optical density at 600 nm of 0.05. The microtiter plate was incubated for 24 hours at 37 ° C. in a LAB COMPANION SI-600R incubating shaker set at 300 rpm. At the end of 24 hours, cell growth was determined by measuring the optical absorbance of a 1 to 10 dilution of the culture at 600 nm in a BIOCHROM LIBRA S22 spectrophotometer. Plasmid retention was determined by serial dilutions and plate counts on LB-agar plates and appropriate selection plates. Selection plates for GEM5 strains bearing plasmids were LB-agar plates supplemented with 20 μg/mL chloramphenicol. Selection plates for GEM8 strains bearing plasmids were minimal medium plates supplemented with 40 g/L glycerol. All plates were incubated at 37° C. for 16 to 24 hours to permit the formation of colonies. Plasmid retention is defined as the number of colonies counted on selection plates divided by the number of colonies on LB-agar plates at the same serial dilution.

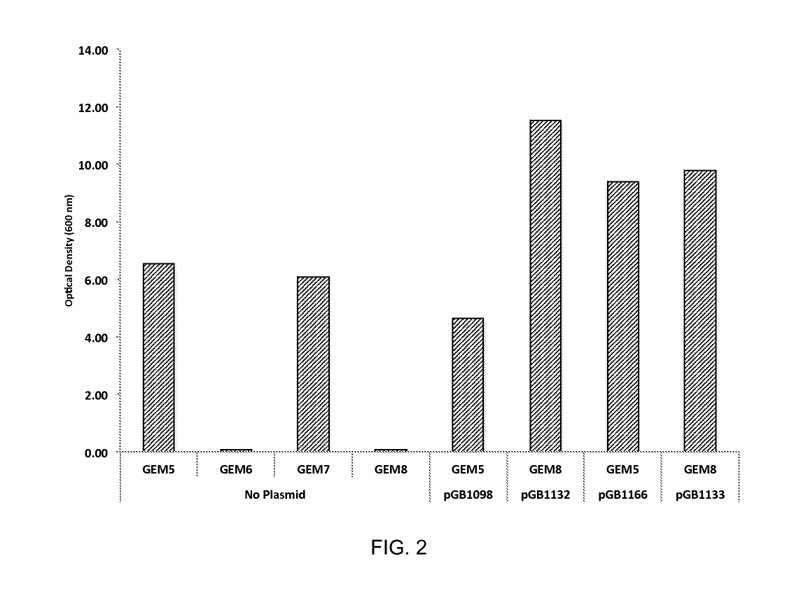

As shown in FIG. 2, deletion of glpK greatly reduces the ability of E. coli to grow aerobically on glycerol as a sole carbon source (compare growth of GEM5 to GEM6 or GEM8). In contrast, deletion of gldA (in GEM7) has little effect on the ability of E. coli to grow aerobically on glycerol (compare GEM5 and GEM7). Deletion of both glpK and gldA from the chromosome can be complemented by plasmids bearing either glpK or gldA under the control of a non-native promoter. Plasmid pGB1132 carrying the gldA gene under control of the alcohol dehydrogenase promoter restores the ability of GEM8 to grow on glycerol under aerobic conditions. Likewise, plasmid pGB1133 carrying the glpK gene under control of the alcohol dehydrogenase promoter restores the ability of GEM8 to grow on glycerol under aerobic conditions.

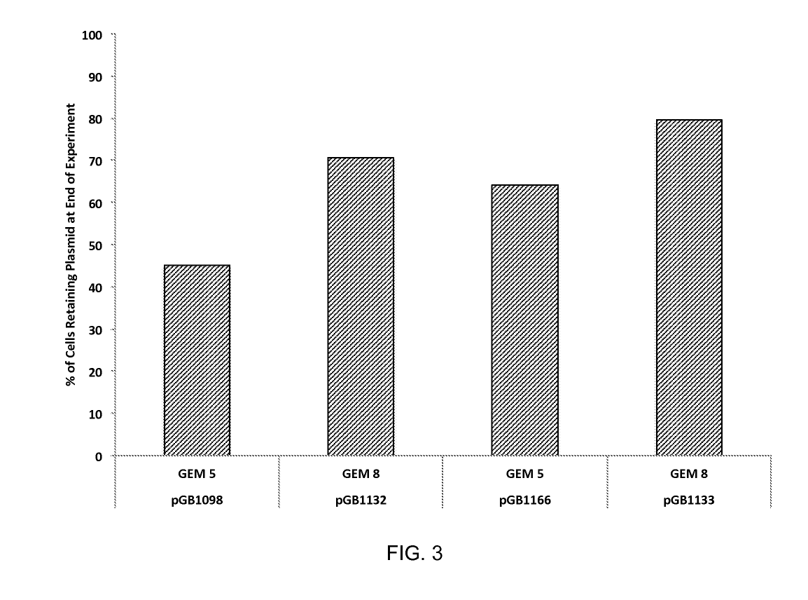

In the absence of antibiotic selective pressure, plasmids pGB1098 and pGB1166 are retained by GEM5 and plasmids pGB1132 and pGB1133 are retained by GEM8 (FIG. 3 and Table 5); however, a higher percentage of GEM8 cells retain plasmids under the conditions used, demonstrating the effectiveness of plasmid stabilization in the absence of either antibiotics in the culture or antibiotic resistance genes on the plasmids.

TABLE 5

| | % of Cells Retaining Plasmid at End of Experiment | | | |
|---|---|---|---|---|
| Strain | pGB1098 | pGB1132 | pGB1166 | pGB1133 |
| GEM5 | 45 | — | 64 | — |
| GEM8 | — | 71 | — | 80 |

### Example 2

### Microaerobic Conditions

This working example demonstrates complementation of E. coli mutants deficient in glycerol dissimilation by plasmids bearing glycerol dissimilation genes but lacking antibiotic resistance genes, under microaerobic conditions. As defined herein, microaerobic conditions have a dissolved oxygen concentration of less than 5%. The following E. coli strains were used in this example: GEM5, GEM6, GEM7, GEM8, GEM5(pGB1098), GEM5(pGB1166), GEM8(pGB1132), and GEM8(pGB1133).

Individual colonies of GEM strains not bearing plasmids were used to inoculate 5 mL of LB medium (10 g/L NaCl, 5 g/L yeast extract, 10 g/L tryptone) in a 14-mL, round-bottom, polypropylene culture tube. Individual colonies of GEM5 or GEM8 strains bearing plasmids were used to inoculate 5 mL of minimal medium supplemented with 20 g/L glycerol and 0.1 M Sorensen's phosphate buffer in a 25-mL Erlenmeyer flask. All cultures of GEM5 strains bearing plasmids were also supplemented with 20 μg/mL chloramphenicol. The 5-mL cultures were incubated for 16 hours at 37 ° C. in a LAB COMPANION SI-600R incubating shaker set at 175 rpm. Cultures grown in LB medium were pelleted by low-speed centrifugation and washed by resuspension in minimal medium.

The 16-hour cultures were used to seed 15-mL cultures of minimal medium supplemented with 20 g/L glycerol and 0.1 M Sorensen's phosphate buffer in a 25-mL Erlenmeyer Flask. All experimental cultures were seeded with sufficient cells to achieve an initial optical density at 600 nm of 0.05. The Erlenmeyer flasks were incubated for 24 hours at 37° C. in a LAB COMPANION SI-600R incubating shaker set at 175 rpm. At the end of 24 hours, cell growth was determined by measuring the optical absorbance of a 1 to 10 dilution of the culture at 600 nm in a BIOCHROM LIBRA S22 spectrophotometer. Plasmid retention was determined by serial dilutions and plate counts on LB-agar plates and appropriate selection plates. Selection plates for GEM5 strains bearing plasmids were LB-agar plates supplemented with 20 μg/mL chloramphenicol. Selection plates for GEM8 strains bearing plasmids were minimal medium plates supplemented with 40 g/L glycerol. All plates were incubated at 37° C. for 16 to 24 hours to permit the formation of colonies. Plasmid retention is defined as the number of colonies counted on selection plates divided by the number of colonies on LB-agar plates at the same serial dilution.

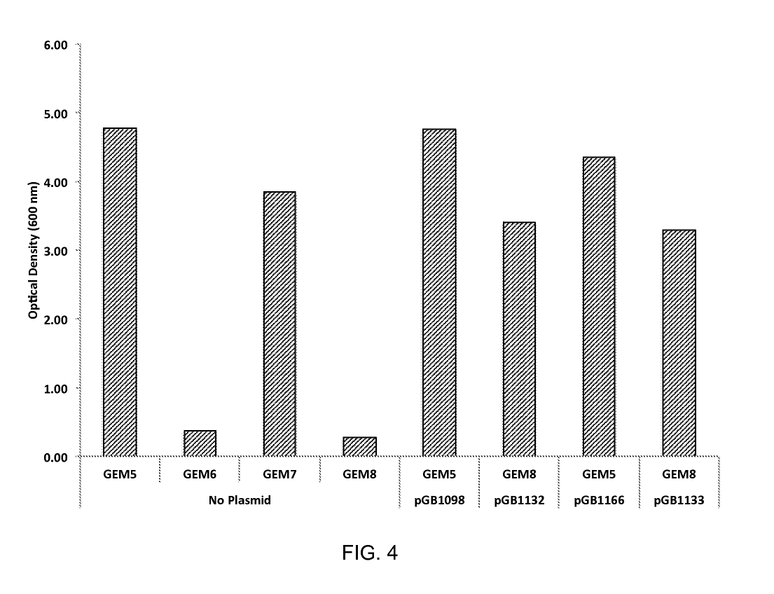

As shown in FIG. 4, deletion of glpK greatly reduces the ability of E. coli to grow under microaerobic conditions on glycerol as a sole carbon source (compare GEM5 to

GEM6 and GEM8). In contrast, deletion of gldA has only a modest effect on the ability of E. coli to grow microaerobically on glycerol (compare GEM5 and GEM7). Deletion of both glpK and gldA from the chromosome can be complemented by plasmids bearing either glpK or gldA under the control of a non-native promoter. Plasmid pGB1132 carrying the gldA gene under control of the alcohol dehydrogenase promoter restores the ability of GEM8 to grow on glycerol under microaerobic conditions. Likewise, plasmid pGB1133 carrying the glpK gene under control of the alcohol dehydrogenase promoter restores the ability of GEM8 to grow on glycerol under microaerobic conditions.

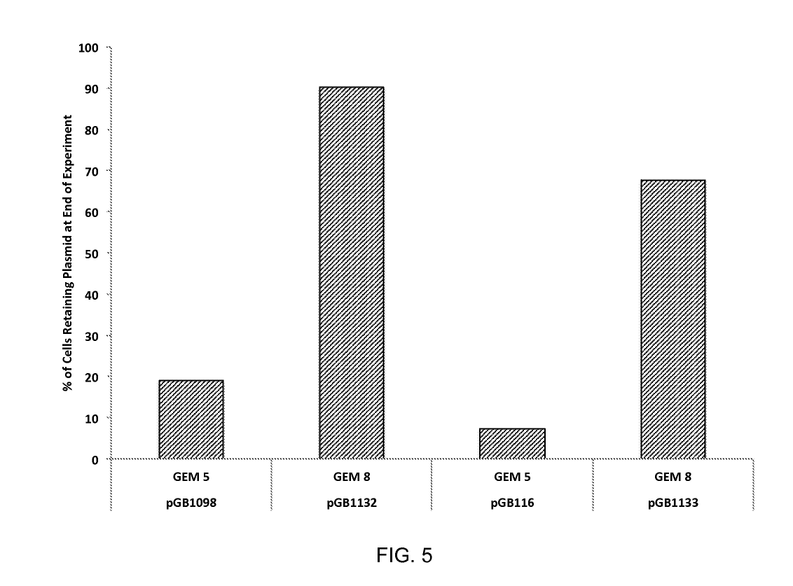

In the absence of antibiotic selective pressure, plasmids pGB1098 and pGB1166 are retained by GEM5 and plasmids pGB1132 and pGB1133 are retained by GEM8 (FIG. 5 and Table 6); however, a higher percentage of GEM8 cells retain either plasmid under the conditions used, demonstrating the effectiveness of plasmid stabilization in the absence of either antibiotics in the culture or antibiotic resistance genes on the plasmids.

TABLE 6

| | % of Cells Retaining Plasmid at End of Experiment | | | |
|---|---|---|---|---|
| Strain | pGB1098 | pGB1132 | pGB1166 | pGB1133 |
| GEM5 | 19 | — | 7 | — |
| GEM8 | — | 90 | — | 68 |

### Example 3

### Anaerobic Conditions

This working example demonstrates complementation of E. coli mutants deficient in glycerol dissimilation by plasmids bearing glycerol dissimilation genes but lacking antibiotic resistance genes, under anaerobic conditions. The following E. coli strains were used in this example: GEM5, GEM6, GEM7, GEM8, GEM5(pGB1098), and GEM8(pGB1132).

Individual colonies of GEM strains not bearing plasmids were used to inoculate 5 mL of LB medium (10 g/L NaCl, 5 g/L yeast extract, 10 g/L tryptone) in a 14-mL, round-bottom, polypropylene tube. Individual colonies of GEM5(pGB1098) or GEM8(pGB1132) strains (bearing plasmids) were used to inoculate 5 mL of minimal medium supplemented with 40 g/L glycerol and 0.1 M Sorensen's phosphate buffer in a 25-mL Erlenmeyer flask. The cultures of GEM5(pGB1098) strain were also supplemented with 20 μg/mL chloramphenicol. The 5-mL cultures were incubated for 16 hours at 37° C. in a LAB COMPANION SI-600R incubating shaker set at 175 rpm. Cultures grown in LB medium were pelleted by low-speed centrifugation and washed by resuspension in minimal medium.

The 16-hour cultures were used to seed 10-mL cultures of minimal medium supplemented with 20 g/L glycerol and 0.1 M Sorensen's phosphate buffer in a Hungate tube (Chemglass, Vineland, N.J.). The septa of the Hungate tubes were pierced with one 6-inch, 18-gauge, stainless steel Luer-Lok, non-coring penetration needle (Popper & Sons, New Hyde Park, N.Y.) attached to a supply of sterile-filtered nitrogen gas and a second, 1.5-inch, 20-gauge stainless steel Luer-Lok hypodermic needle (Becton Dickinson, Franklin Lakes, N.J.) attached to a 0.45 μm, 30 mm diameter syringe filter. All experimental cultures were seeded with sufficient cells to achieve an initial optical density at 600 nm of 0.05. All cultures were continuously sparged with sterile nitrogen gas while being incubated for 48 hours at 37° C. in a water bath. At the end of 24 and 48 hours, cell growth was determined by measuring the optical absorbance of a 1 to 10 dilution of the culture at 600 nm in a BIOCHROM LIBRA S22 spectrophotometer. Plasmid retention at 48 hours was determined by serial dilutions and plate counts on LB-agar plates and appropriate selection plates. Selection plates for GEM5 strains bearing plasmids were LB-agar plates supplemented with 20 μg/mL chloramphenicol. Selection plates for GEM8 strains bearing plasmids were minimal medium plates supplemented with 40 g/L glycerol. All plates were incubated at 37° C. for 16 to 24 hours to permit the formation of colonies. Plasmid retention is defined as the number of colonies counted on selection plates divided by the number of colonies on LB-agar plates at the same serial dilution.

As shown in FIG. 6, deletion of glpK modestly reduces the ability of E. coli to grow under anaerobic conditions on glycerol as a sole carbon source (compare GEM5 to GEM6). In contrast, deletion of gldA has a stronger effect on the ability of E. coli to grow anaerobically on glycerol (compare GEM5 and GEM7). These results demonstrate that the gldA gene product plays a more important role than the glpK gene product under anaerobic conditions. Deletion of both glpK and glpD reduces cell growth to essentially no growth (compare GEM5 to GEM8). Deletion of both glpK and gldA from the chromosome can be complemented by plasmids bearing either glpK or gldA under the control of a non-native promoter. Plasmid pGB1132 carrying the gldA gene under control of the alcohol dehydrogenase promoter restores the ability of GEM8 to grow on glycerol under anaerobic conditions. Likewise, plasmid pGB1133 carrying the glpK gene under control of the alcohol dehydrogenase promoter restores the ability of GEM8 to grow on glycerol under anaerobic conditions.

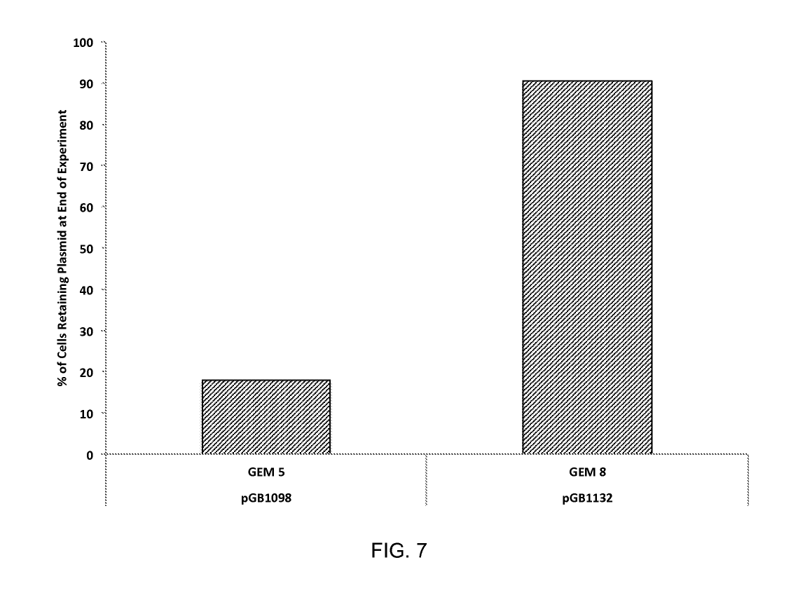

In the absence of antibiotic selective pressure, plasmid pGB1098 is retained by GEM5 and plasmid pGB1132 is retained by GEM8 (FIG. 7 and Table 7); however, a higher percentage of GEM8 cells retain plasmid under the conditions used, demonstrating the effectiveness of plasmid stabilization in the absence of either antibiotics in the culture or antibiotic resistance genes on the plasmids.

TABLE 7

% of Cells Retaining Plasmid at End of Experiment

| Strain | pGB1098 | pGB1132 |
|---|---|---|
| GEM5 | 18 | — |
| GEM8 | — | 91 |

### Example 4

### Plasmid Stabilization During Ethanol Production

This working example demonstrates the production of glycerol from ethanol by E. coli strains GEM5(pGB1098) and GEM8(pGB1132) under microaerobic conditions in the absence of antibiotic resistance genes.

Individual colonies of GEM5(pGB1098) or GEM8(pGB1132) strains were used to inoculate 40 mL of minimal medium supplemented with 40 g/L glycerol and 0.1 M Sorensen's phosphate buffer in a 250-mL baffled Erlenmeyer flask. GEM5(pGB1098) was also supplemented with 20 μg/mL chloramphenicol. The 40-mL cultures were incubated for 16 hours at 37° C. in a LAB COMPANION SI-600R incubating shaker set at 175 rpm.

The 16-hour cultures were used to seed 400-mL cultures of minimal medium supplemented with 60 g/L glycerol in a 0.5-L working volume fermentor (Ward's Natural Science, Rochester, N.Y.) with independent control of temperature, pH, and stirrer speed. All experimental cultures were seeded with sufficient cells to achieve an initial optical density at 600 nm of 0.1. One culture consisted of GEM5(pGB1098) supplemented with 20 μg/mL chloramphenicol. A second culture consisted of GEM(pGB1098) without chloramphenicol supplementation. A third culture consisted of GEM8(pGB1132) without chloramphenicol supplementation. Temperature was maintained at 37° C. pH was maintained at 7.0 using 5 N NaOH. The stirrer speed was maintained at 200 rpm. The cultures were aerated at 10 mL/min using air, sufficient to achieve a k_{L}a of 60h⁻¹. 5-mL samples were withdrawn from the cultures at 0-, 24-, 48- and 72-hour time points. The samples were used to measure cell growth, plasmid retention, glycerol consumption, and metabolite production. Cell growth was determined by measuring the optical absorbance of a 1 to 10 dilution of the culture at 600 nm in a BIOCHROM LIBRA S22 spectrophotometer. Plasmid retention was determined by serial dilutions and plate counts on LB-agar plates and appropriate selection plates. Selection plates for GEM5 strains bearing plasmids were LB-agar plates supplemented with 20 μg/mL chloramphenicol. Selection plates for GEM8 strains bearing plasmids were minimal medium plates supplemented with 40 g/L glycerol. All plates were incubated at 37° C. for 16 to 24 hours to permit the formation of colonies. Plasmid retention is defined as the number of colonies counted on selection plates divided by the number of colonies on LB-agar plates at the same serial dilution.

Glycerol consumption and metabolite production were quantified by HPLC analysis. All samples were filtered through a 0.22 μm polyvinylidene fluoride syringe filter (Millipore) prior to HPLC analysis. Routinely, 10-μL of filtered fermentation medium was injected onto an HPLC (LC-10AD vp, Shimadzu, Kyoto, Japan) fitted with a REZEX ROA-ORGANIC ACID H⁺ (8%) 150×7.8 mm column (Phenomenex, Torrance, CA) at 65° C. with a mobile phase of 2.5 mM H₂SO₄ operated under isocratic conditions at a flow rate of 0.6 mL/minute. Metabolites were detected via a refractive index detector (RID-10A, Shimadzu).

The results of the experiment demonstrate that plasmids bearing genes encoding enzymes involved in glycerol dissimilation can be stably maintained in host cells lacking genes encoding glycerol dissimilation when cultured on glycerol as a sole carbon source; neither antibiotics nor antibiotic resistance genes (selectable markers) are required.

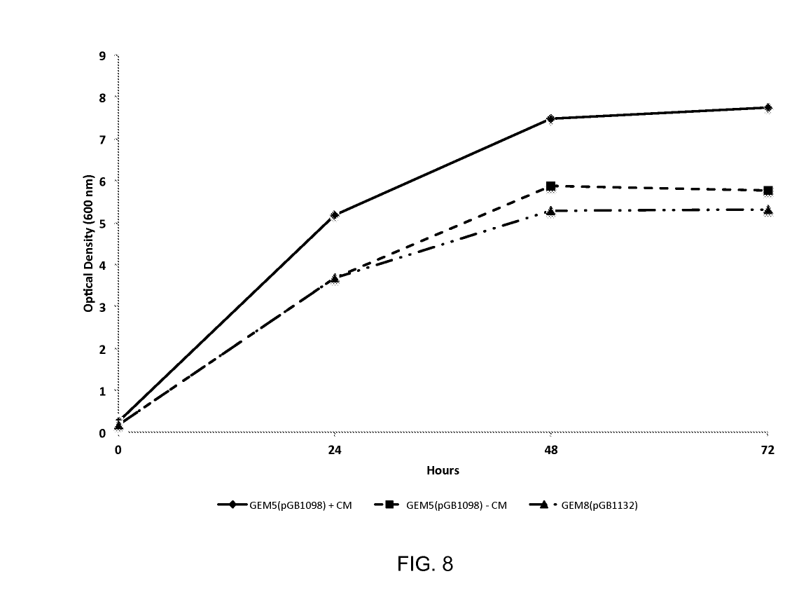

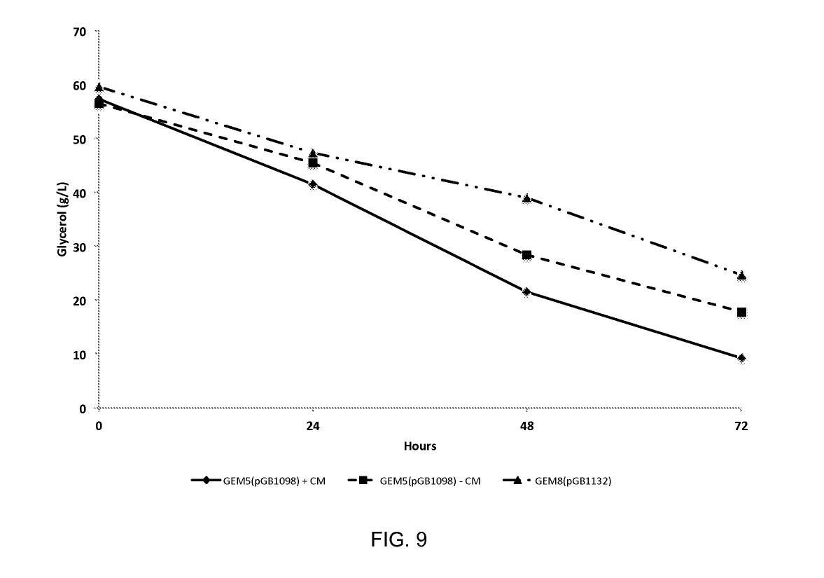

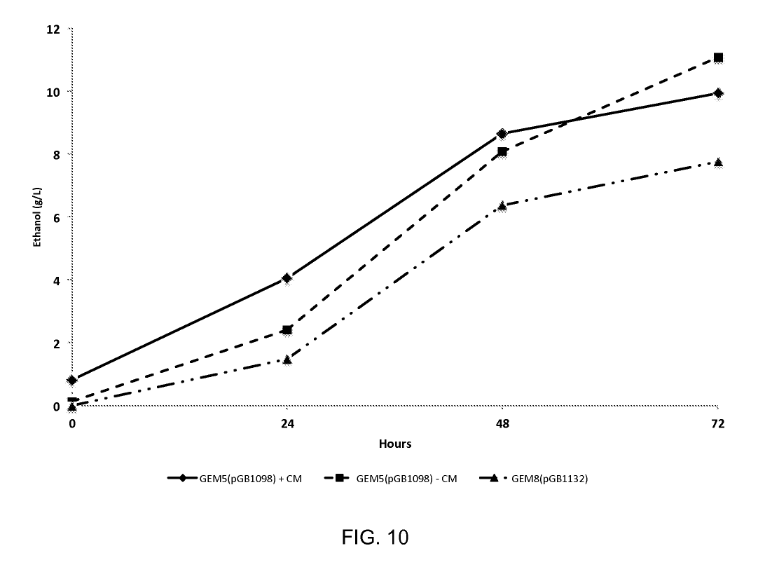

GEM5(pGB1098), with or without antibiotics present in the medium, and GEM8(pGB1132) show similar growth profiles (FIG. 8), rates of glycerol consumption (FIG. 9), and ethanol production (FIG. 10). Surprisingly, plasmid retention is higher in GEM8(pGB1132)—63%—than in GEM5 with or without antibiotics (46% and 18%, respectively).

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While the foregoing is directed to embodiments of the present invention, other and further embodiments of the invention may be devised without departing from the basic scope thereof, and the scope thereof is determined by the claims that follow.

1. An isolated transformed microbial host cell comprising:
a. A deletion, a disruption or a mutation that reduces or eliminates the activity of one or more chromosomal genes encoding one or more enzymes of the glycerol dissimilation pathway; and
b. A plasmid comprising at least one of the one or more genes encoding one or more enzymes of the glycerol dissimilation pathway operably linked to a promoter, said plasmid lacking an antibiotic resistance gene,
wherein the plasmid is stably maintained in the isolated transformed microbial host cell when grown in the presence of glycerol.

2. An isolated transformed microbial host cell according to claim 1, wherein the glycerol is the sole carbon source provided to the cell.

3. An isolated transformed microbial host cell according to claim 1, wherein the one or more genes of the glycerol dissimilation pathway encoded by the plasmid is a glycerol dehydrogenase gene and the chromosomal glycerol dehydrogenase gene is deleted, disrupted or mutated to reduce or eliminate its activity.

4. An isolated transformed microbial host cell according to claim 1, wherein the one or more genes of the glycerol dissimilation pathway encoded by the plasmid is a glycerol kinase gene and the chromosomal glycerol kinase gene is deleted, disrupted or mutated to reduce or eliminate its activity.

5. An isolated transformed microbial host cell according to claim 1, wherein the one or more genes of the glycerol dissimilation pathway encoded by the plasmid is a glycerol dehydrogenase and the chromosomal glycerol kinase gene is deleted, disrupted or mutated to reduce or eliminate its activity.

6. An isolated transformed microbial host cell according to claim 1, wherein the one or more genes of the glycerol dissimilation pathway encoded by the plasmid is a glycerol kinase and the chromosomal glycerol dehydrogenase gene is deleted, disrupted or mutated to reduce or eliminate its activity.

7. An isolated transformed microbial host cell according to claim 1, wherein the one or more genes of the glycerol dissimilation pathway encoded by the plasmid is a glycerol dehydrogenase gene and the chromosomal genes encoding glycerol dehydrogenase and glycerol kinase are deleted, disrupted or mutated to reduce or eliminate activity.

8. An isolated transformed microbial host cell according to claim 1, wherein the one or more genes of the glycerol dissimilation pathway encoded by the plasmid is a glycerol kinase gene and the chromosomal genes encoding glycerol dehydrogenase and glycerol kinase are deleted, disrupted or mutated to reduce or eliminate activity.

9. A method of maintaining a plasmid in a transformed microbial host cell comprising the step of culturing an isolated transformed host cell of claim 1 in the presence of glycerol under conditions sufficient to permit said cell to grow.

10. The method of claim 9, wherein the glycerol is the sole carbon source provided to the cell.

11. The method of claim 9, wherein the growth conditions are anaerobic and the isolated transformed microbial host cell comprises a deletion, disruption or mutation of the chromosomal gene encoding glycerol dehydrogenase gene to reduce or eliminate its activity and the plasmid comprises a glycerol dehydrogenase gene operably linked to a promoter.

12. The method of claim 9, wherein the growth conditions are anaerobic and the isolated transformed microbial host cell comprises a deletion, disruption or mutation of the chromosomal gene encoding glycerol dehydrogenase to reduce or eliminate its activity, a deletion, disruption or mutation of the chromosomal gene encoding glycerol kinase to reduce or eliminate its activity, and the plasmid comprises a glycerol dehydrogenase gene operably linked to a promoter.

13. The method of claim 9, wherein the growth conditions are microaerobic and the isolated transformed microbial host cell comprises a deletion, disruption or mutation of the chromosomal gene encoding glycerol dehydrogenase gene to reduce or eliminate its activity and the plasmid comprises a glycerol dehydrogenase gene operably linked to a promoter.

14. The method of claim 9, wherein the growth conditions are microaerobic and the isolated transformed microbial host cell comprises a deletion, disruption or mutation of the chromosomal gene encoding glycerol dehydrogenase to reduce or eliminate its activity, a deletion, disruption or mutation of the chromosomal gene encoding glycerol kinase to reduce or eliminate its activity, and the plasmid comprises a glycerol dehydrogenase gene operably linked to a promoter.

15. The method of claim 9, wherein the growth conditions are aerobic and the isolated transformed microbial host cell comprises a deletion, disruption or mutation of the chromosomal gene encoding glycerol kinase to reduce or eliminate its activity, and the plasmid comprises a glycerol kinase gene operably linked to a promoter. 16-17. (canceled)

18. An isolated transformed microbial host cell comprising:
a. a deletion, disruption or mutation that reduces or eliminates the activity in two or more chromosomal genes encoding two or more enzymes of the glycerol dissimilation pathway; and
b. two or more plasmids wherein each plasmid encodes one or more enzymes of the glycerol dissimilation pathway operably linked to a promoter, said plasmids lacking an antibiotic resistance gene,
wherein the two or more plasmids are stably maintained in the isolated transformed microbial host cell when grown in the presence of glycerol.

19. An isolated transformed host cell according to claim 18, wherein the glycerol is the sole carbon source provided to the cell.

20. An isolated transformed host cell according to claim 18, wherein the gene of the glycerol dissimilation pathway encoded by the first plasmid is a glycerol kinase gene and the gene of the glycerol dissimilation pathway encoded by the second plasmid is a glycerol-3-phosphate dehydrogenase gene; and the chromosomal glycerol kinase gene and glycerol-3-phosphate gene are deleted, disrupted or mutated to reduce or eliminate its activity.

21. An isolated transformed host cell according to claim 18, wherein the gene of the glycerol dissimilation pathway encoded by the first plasmid is a glycerol dehydrogenase gene and the gene of the glycerol dissimilation pathway encoded by the second plasmid is one or more subunits of a dihydroxyacetone kinase operon; and the chromosomal glycerol dehydrogenase gene and the one or more subunits of the chromosomal dihydroxyacetone kinase operon are deleted, disrupted or mutated to reduce or eliminate its activity.

22. A method of maintaining two or more plasmids in a transformed microbial host cell comprising the step of culturing an isolated transformed host cell of claim 18 in the presence of glycerol under conditions sufficient to permit said cell to grow.

23. The method of claim 22, wherein the glycerol is the sole carbon source provided to the cell.

24. The method of claim 22, wherein the growth conditions are anaerobic or microaerobic and the isolated transformed microbial host cell comprises a deletion, disruption or mutation of the glycerol dehydrogenase gene and one or more subunits of the dihydroxyacetone kinase operon to reduce or eliminate the activities of these enzymes; a first plasmid comprising a glycerol dehydrogenase gene operably linked to a promoter; and, a second plasmid comprising one or more genes encoding subunits of dihydroxyacetone kinase operably linked to a promoter.

25. The method of claim 22, wherein the growth conditions are aerobic or microaerobic and the isolated transformed host cell comprises a deletion, disruption or mutation of the glycerol kinase gene and the glycerol-3-phosphate dehydrogenase gene to reduce or eliminate activities of these enzymes; a first plasmid comprising a glycerol kinase gene operably linked to a promoter; and, a second plasmid comprising a glycerol-3-phosphate dehydrogenase gene operably linked to a promoter. 26-30. (canceled)

31. An isolated transformed microbial host cell according to claim 1 or claim 16, wherein the microbial host cell is selected from the genus Escherichia, Salmonella, Enterobacter, Klebsiella, Citrobacter, and Bacillus.
